(19) United States
(12) Patent Application Publication
(10) Pub. No.: US 2017/0215844 A1
(43) Pub. Date: Aug. 3, 2017

(54) SYSTEMS FOR ULTRASOUND IMAGING

(71) Applicant: General Electric Company, Schenectady, NY (US)

(72) Inventors: Xiaodong Han, Shanghai (CN); Menachem Halmann, Wauwatosa, WI (US); William John Jaworski, Wauwatosa, WI (US); Zhenyu Liu, Wuxi (CN); Gang Cheng, Shanghai (CN); Dongqing Chen, Wauwatosa, WI (US)

(21) Appl. No.: 15/515,248

(22) PCT Filed: Sep. 15, 2015
(86) PCT No.: PCT/US2015/050150

§ 371 Date: Mar. 29, 2017

(30) Foreign Application Priority Data
Sep. 30, 2014 (CN) 201410519494.4

(51) Int. Cl. A61B 8/00; A61B 8/06; A61B 8/08

(52) U.S. Cl. CPC A61B 8/4281; A61B 8/40; A61B 8/4461; A61B 8/4245; A61B 8/488; A61B 8/06; A61B 8/4218; A61B 8/5246

(57) ABSTRACT

A system is provided. The system includes a tank, a holder, a transducer array and an actuator. The tank is configured to contain fluid and allow a hand to be immersed in the fluid. The holder is located in the tank and configured to hold the hand. The transducer array is positioned adjacent to the tank and operable in at least two imaging modes. The actuator is coupled to the transducer array and configured to move the transducer array in the at least two imaging modes. And an ultrasound system is also provided, which includes a processing unit for generating images in the different imaging modes.

### CROSS-REFERENCE TO RELATED APPLICATIONS

This is a national stage application under 35 U.S.C. §371(c) of PCT Application No. PCT/US2015/050150, filed on Sep. 15, 2015, which claims priority to Chinese Application No. 201410519494.4, filed on Sep. 30, 2014, the disclosures of which are incorporated herein by reference in their entireties.

### BACKGROUND

Embodiments of the disclosure relate generally to ultrasound systems, and more particularly to ultrasound systems for automated ultrasound based detection, quantification, and tracking of musculoskeletal pathologies.

Arthritis in finger joints is one of the most chronic diseases in the U.S. Currently, the finger joints are scanned manually by an ultrasound probe which is held by doctors. Blood flow through the finger joints can indicate the arthritis states. However, the blood flow is quite sensitive to environments, such as pressure of the ultrasound probe on the finger joints, environment temperature. It is very difficult for less-experienced doctors to intelligently, objectively and accurately diagnose the arthritis, monitor scanning progress and evaluate treatment prognosis through current ultrasound scanning and ultrasound images.

It is desirable to provide a solution to address at least one of the above-mentioned problems.

### BRIEF DESCRIPTION

A system is provided. The system includes a tank, a holder, a transducer array and an actuator. The tank is configured to contain fluid and allow a hand to be immersed in the fluid. The holder is located in the tank and configured to hold the hand. The transducer array is positioned adjacent to the tank and operable in at least two imaging modes. The actuator is coupled to the transducer array and configured to move the transducer array in the at least two imaging modes.

A system is provided. The system includes a tank, a holder, a transducer array and processing unit. The tank is configured to contain fluid and allow a hand to be immersed in the fluid. The holder is located in the tank and configured to hold the hand. The transducer array is positioned adjacent to the tank and operable in a first imaging mode and a second imaging mode. The transducer array is configured to scan the hand in the first imaging mode and scan a plurality of finger joints of the hand in the second imaging mode. The processing unit is coupled to the transducer array and configured to control the transducer array in the first imaging mode and the second imaging mode. The processing unit is configured to generate ultrasound images of the hand in the first imaging mode and generate images of the plurality of finger joints in the second imaging mode.

### DRAWINGS

These and other features and aspects of embodiments of the present invention will become better understood when the following detailed description is read with reference to the accompanying drawings in which like characters represent like parts throughout the drawings, wherein:

FIG. 1 is a schematic view of an ultrasound system according to one embodiment;

FIG. 2 is a schematic side view of a scanning apparatus of the ultrasound system of FIG. 1;

FIG. 3 is a schematic view of a holder of the ultrasound system according to another embodiment;

FIG. 4 is 2D ultrasound images of slices of the hand according to an embodiment; and

FIG. 5 is an image of a finger joint of the hand according to an embodiment.

### DETAILED DESCRIPTION

Unless defined otherwise, technical and scientific terms used herein have the same meaning as is commonly understood by one of ordinary skill in the art to which this invention belongs. The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced items. Moreover, the terms “coupled” and “connected” are not intended to distinguish between a direct or indirect coupling/connection between two components. Rather, such components may be directly or indirectly coupled/connected unless otherwise indicated.

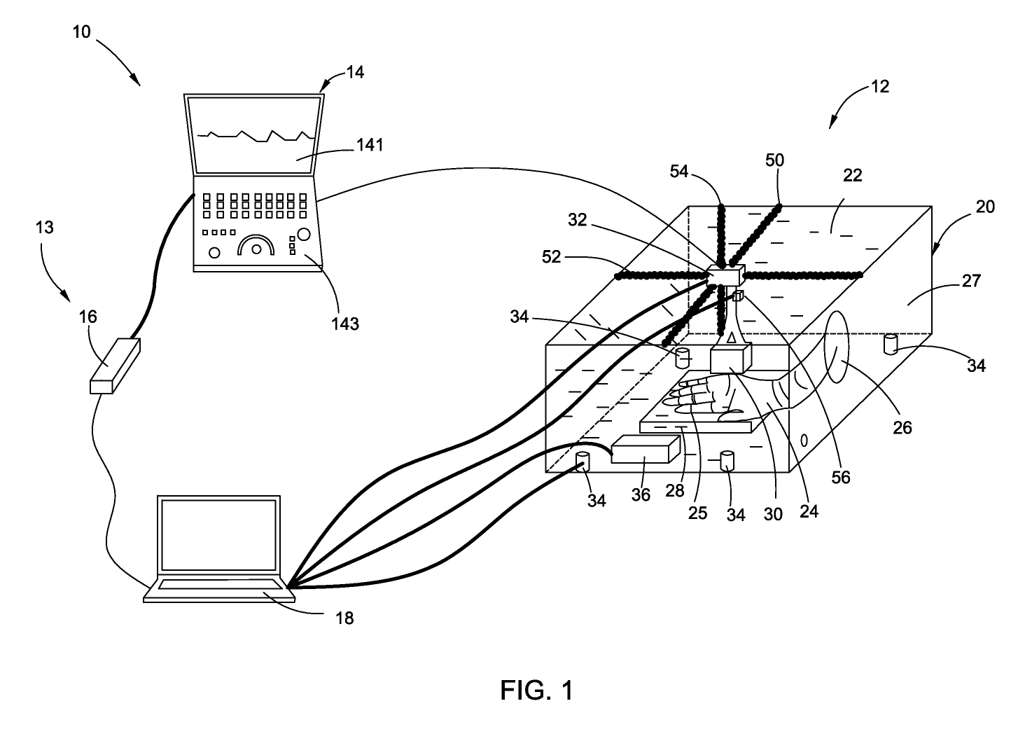

FIG. 1 illustrates a schematic view of an ultrasound system 10 according to one embodiment. The ultrasound system 10 is configured to acquire ultrasound images of hands of patients for a rheumatoid arthritis examination. The ultrasound system 10 may be employed to facilitate automated detection, quantification and tracking of rheumatoid arthritis using the ultrasound images. The ultrasound system 10 includes a scanning apparatus 12 and a processing unit 13. The processing unit 13 includes an imaging device 14, a screen grabbing device 16 and a controller 18.

The scanning apparatus 12 is configured to sweep a hand 24 of a patient accommodated therein through ultrasound. The scanning apparatus 12 includes a tank 20, a holder 28, a transducer array 30 and an actuator 32. The tank 20 is configured to contain fluid 22 and allow the hand 24 to be immersed in the fluid 22. The fluid 22 servers as an acoustic coupling between the hand 24 and the transducer array 30. In an exemplary embodiment, the fluid 22 includes water allowing ultrasound to pass through. In another embodiment, the fluid 22 may include other forms of a fluid, such as other forms of a liquid, gel or the like, which serve as an acoustic coupling between the hand 24 and the transducer array 30.

The tank 20 is configured to contain the fluid 22. And the tank 20 is sized and configured to receive the hand 24 such that the hand 24 as immersed within fluid 22. The tank 20 includes a hole or an open 26 on a side surface 27 thereof which is configured to allow the hand 24 into the tank 20. The holder 28 is located in the tank 20 and configured to hold the hand 24. The holder 28 includes structures within the fluid 22 which are shaped, sized and/or otherwise configured to retain the hand 24 stationary in the fluid 22 during scanning

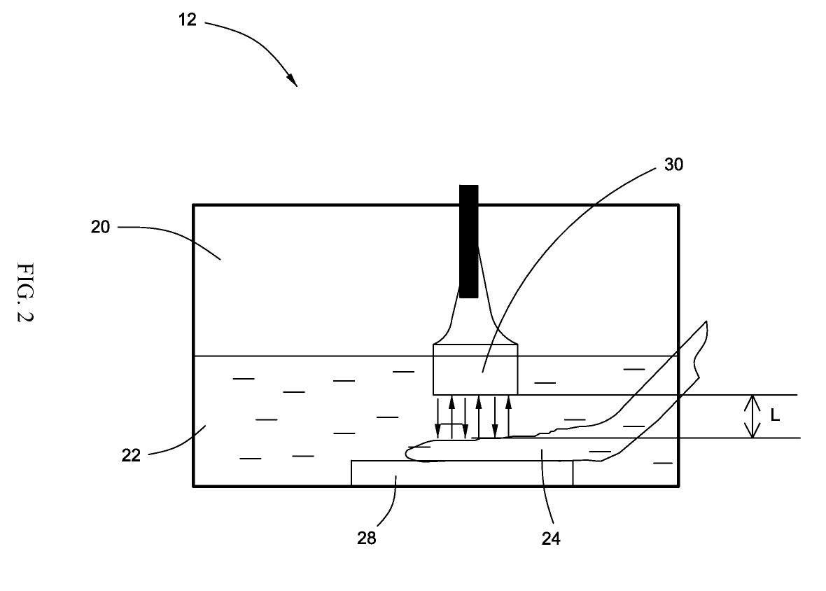

The transducer array 30 is positioned adjacent to the tank 20. The transducer array 30 is acoustic coupled with the hand 24 through the fluid 22. At least part of the transducer array 30 is immersed in the fluid 22 and the transducer array 30 is apart from the hand 24 with a gap L which is about 5 mm to 10 mm for example as shown in FIG. 2. The transducer array 30 is configured to emit ultrasound signals. The transducer array 30 includes an array of transducers that emit the ultrasound signals. In one embodiment, the transducer array 30 includes piezoelectric elements (not shown) that fire ultrasound pulses. The ultrasound signals are transmitted through the fluid 22 to the hand 24. The transducer array 30 is also configured to receive echo signals from the hand 24 which are generated based on the ultrasound signals. The hand 24 is not pressed by the transducer array 30 because of the non-contact there between so as to avoid the detection of rheumatoid arthritis being influenced by the pressure of the transducer array 30. Accordingly, accuracy of the detection is improved.

The transducer array 30 is operable in at least two imaging modes. The imaging modes are different. In one embodiment, a first imaging mode of the imaging modes is a B-mode in which the entire hand 24 is scanned by the transducer array 30. The transducer array 30 scans the hand 24 slice by slice. Adjacent scanning slices are apart about 0.1 mm to 0.5 mm in one embodiment. A second imaging mode of the imaging modes is a Power Doppler Imaging (PDI) mode or a high resolution PDI mode in which joints 25 of the hand 24 is scanned. The joints 25 include metacarpophalangeal (MCP), proximal interphalangeal (PIP) and distal interphalangeal (DIP) joints of fingers, digits or phalanges of the hand 24. For purposes of this disclosure, the term “fingers” includes a person's thumb as well as the remaining digits of a hand. In the second imaging mode, the transducer array 30 is moved to the locations of the joints 25 and scans each joint 25 slice by slice. The transducer array 30 stops for at least a cardiac cycle which is about 1.5 second to 2 seconds for scanning each slice of the joints 25. In one embodiment, the slices in scanning the joints 25 have same positions with the corresponding slices in scanning the hand 24. In another embodiment, the distance between adjacent scanning slices in scanning the joints 25 may be different from that in scanning the entire hand 24 according to particular applications. That is to say, step-length of the transducer array 30 in scanning the joints 25 may be different from that in scanning the entire hand 24. In another embodiment, the imaging modes may include three or more than three modes. The transducer array 30 can be switched to different imaging modes.

In one embodiment, the transducer array 30 is a high frequency probe emitting high frequency ultrasound which has a frequency about 15 MHz or more than 15 MHz for example. In one embodiment, the frequency of the ultrasound may be about 18 MHz, 20 MHz or 22 MHz. The hand 24 and the joints 25 of the hand 24 can be imaged clearly in the high frequency ultrasound.

Continuing to refer to FIG. 1, the actuator 32 is coupled to the transducer array 30 and configured to move the transducer array 30 in the different imaging modes. The actuator 32 is configured to selectively position the transducer array 30 with respect to the hand 24 in response to control signals received from the controller 18. In one embodiment, the actuator 32 may include one or more motors, such as stepper motors, which are powered by a power source (not shown) and drove by motor drivers (not shown) to selectively position the transducer array 20.

In one embodiment, multiple guide rails 50, 52 and 54 are connected with the transducer array 30. The guide rails 50, 52 and 54 are configured to support the transducer array 30 in a way to allow the transducer array to slide or move along the guide rails 50, 52 and 54. The actuator 32 drives the transducer array 30 to move along the guide rails 50, 52 and 54. In one embodiment, the guide rails 50, 52 and 54 include a longitudinal rail 50, a transverse rail 52 perpendicular to the longitudinal rail 50 and a vertical rail 54 perpendicular to the longitudinal rail 50 and the transverse rail 52. The transducer array 30 may be moved along the longitudinal rail 50, the transverse rail 52 and the vertical rail 54. Accordingly, the transducer array 30 may be moved in a longitudinal direction, in a transverse direction and a vertical direction.

In one embodiment, the transducer array 30 is formed in a probe, the probe having a length of 3 cm for example. The transducer array 30 is moved along the transverse rail 52 to sweep an entire transverse slice of the hand 24 and moved along the longitudinal rail 50 to scan the entire hand 24 slice by slice. The transducer array 30 is also moved along the vertical rail 54 to adjust the distance between the transducer array 30 and the hand 24. In another embodiment, the transducer array 30 is formed in a long probe with 9cm length for example. The transducer array 30 can sweep the entire transverse slice of the hand 24 at a time. The transducer array 30 may be moved along the longitudinal rail 50 and the vertical rail 54 during scanning The transverse rail 52 may be omitted in this embodiment. In still another embodiment, one or more than three guide rails may be employed. For example, the transducer array 30 is tilted along a tilted rail (not shown) or circled along a circle rail (not shown) to sweep three-dimensional (3D) area of the hand 24 including top area and side area of the hand 24.

At least one position sensor 56 is provided to detect positions of the transducer array 30. In one embodiment, the position sensors 56 detect 3D positions of the transducer array 30. The position sensors 56 detect the positions of the transducer array 30 when scanning each slice of the hand 24. Accordingly, each slice of the hand 24 corresponds to a position of the transducer array 30. Signals from the position sensor 56 are communicated to the controller 18.

One or more temperature sensors 34 are provided to detect temperature of the fluid 22. In one embodiment, the temperature sensors 34 are submersed within the fluid 22. In another embodiment, the temperature sensors 34 are external to the fluid 22. The temperature sensors 34 sense the temperature of the fluid 22 contained within the tank 20 and output signals indicating the temperature of the fluid 22. The signals from the temperature sensors 34 are communicated to the controller 18. In one embodiment, the temperature sensors 34 may include a thermocouple sensor, an infrared radiation thermometer, a thermistor thermometer, a resistance temperature detector (RTD), an IC (integrated circuit) temperature sensor or any other types of temperature sensors.

A temperature changing device 36 is provided to regulate the temperature of the fluid 22. The temperature changing device 36 is operable to warm or cool the fluid 22 to regulate the temperature of the fluid 22 at a desirable value, for example, 20° C., 30° C., 40° C. In one embodiment, the temperature changing device 36 is submersed within the fluid 22. In another embodiment, the temperature changing device 36 is external to the tank 20. In one embodiment, the temperature changing device 36 includes an electric heater. In another embodiment, the temperature changing device 36 includes other types of heating elements. In one embodiment, the temperature sensors 34 and the temperature changing device 36 are omitted.

The imaging device 14 is coupled to the transducer array 30 and configured to generate and display ultrasound images of the hand 24. The imaging device 14 receives the echo signals from the transducer array 30 and generates multiple 2D images of the slices of the hand 24 based on the echo signals. The imaging device 14 is also operable to operate in different imaging modes, such as B-mode, PDI mode or high resolution PDI mode. In one embodiment, the imaging device 14 works in the B-mode while the transducer array 30 operates in the B-mode to generate the ultrasound images of the hand 24. The ultrasound images of the hand 24 herein include 2D B-mode ultrasound images of the slices of the entire hand 24. The imaging device 14 may be switched to operate in the PDI mode or the high resolution PDI mode while the transducer array 30 operates in the same mode to generate PDI images or high resolution PDI images of the finger joints 25 which show blood flow condition at the finger joints 25.

In one embodiment, the imaging device 14 includes a programmed unit with programmed logic or a programmed algorithm to generate the ultrasound images in the different imaging modes. The programmed algorithm includes different imaging algorithm for the different imaging modes. In one embodiment, the imaging device 14 includes a screen 141, such as LCD, monitor, LED displayer or the like, for displaying the images. In one embodiment, the imaging device 14 also includes an input device 143, such as keyboard, mouse or the like, for inputting instructions. For example, the imaging device 14 may receive instructions from the input device 143 to switch the transducer array 30 to the different imaging modes.

The screen grabbing device 16 is coupled to the imaging device 14 and configured to grab the screen 141 of the imaging device 14 to get the ultrasound images from the screen 141. The screen grabbing device 16 grabs the 2D ultrasound images of the slices of the entire hand 24 from the screen 141 of the imaging device 14 in real-time with displaying in the imaging device 14 during scanning As such, the screen grabbing device 16 is further configured to communicate the grabbed images to the controller 18. The screen grabbing device 16, in one embodiment, may be connected to an output port of the imaging device 14, such as HDMI (High-Definition Multimedia interface). In another embodiment, the screen grabbing device 16 may be connected with the imaging device 14 in a wireless manner

The controller 18 is coupled to the transducer array 30, the actuator 32, the position sensor 56, the temperature sensors 34 and the temperature changing device 36. The controller 18 receives signals from the position sensor 56 and the temperature sensors 34, and includes a processing unit that controls operation of the transducer array 30, the actuator 32 and the temperature changing device 36. And the controller 18 is also coupled to the screen grabbing device 16 and configured to receive the ultrasound images therefrom.

The controller 18 outputs control signals to the temperature changing device 36 based on the detected temperature of the fluid 22 to control the operation of the temperature changing device 36. The temperature sensors 34 are operative to detect the temperature of the fluid 22 and generate electrical signals indicating the detected temperature of the fluid 22. The electrical signals are received by the controller 18, and the controller 18 generates the control signals based on the electrical signals. The temperature changing device 36 is operative to warm or cool the fluid 22 in response to the control signals. During scanning the joints 25 of the hand 24, the temperature sensors 34, the temperature changing device 36 and the controller 18 operate cooperatively to make sure the temperature of the fluid 22 is constant until all the joints 25 of the hand 24 are scanned. The temperature of the fluid 22 is adjusted between 20° C. to 40° C. in one embodiment. The finger joints 25 are scanned at a constant temperature to detect blood flow through all the slices of the finger joints 25 at the same temperature so as to avoid temperature influence for the bloodstream. In one embodiment, all the joints 25 are scanned at a constant temperature (for example room temperature about 20° C.) of the fluid 22 and after that all the joints 25 are scanned at another constant temperature(s) (for example a warmer temperature about 30° C. to 40° C.) of the fluid 22. The same location of the joint 25 is scanned at two or more than two different temperatures of the fluid 22 to detect the bloodstream through the joints 25 at different temperatures so as to facilitate assessing need for treatment and determining suitable treatment options for the rheumatoid arthritis.

The controller 18 controls the operation and/or positioning of the transducer array 30. In one embodiment, the controller 18 controls the actuator 32 to move the transducer array 30 to sweep the entire hand 24 in the first imaging mode, such as the B-mode. The position sensor 56 senses the positions of the transducer array 30 in real-time and the controller 18 receives and records the signals indicating the positions from the position sensor 56 real-timely. Meanwhile, the imaging device 14 images the 2D ultrasound images of each slice of the entire hand 24 and the screen grabbing device 16 grabs the ultrasound images from the imaging device 14 real-timely. The controller 18 is coupled to the screen grabbing device 16 and configured to receive the ultrasound images therefrom. Each 2D ultrasound image corresponds to a corresponding position of the transducer array 30. In one embodiment, the controller 18 records the 2D ultrasound images with the corresponding positions of the transducer array 30.

In the illustrated embodiment, the controller 18 is further configured to identify locations of the finger joints 25 of the hand 24 from the ultrasound images. In one embodiment, the controller 18 reconstructs a 3D ultrasound image of the hand 24 based on the 2D ultrasound images from the imaging device 14. In one embodiment, the controller 18 identifies the finger joints 25 from the 3D ultrasound image of the hand 24. For example, the controller 18 identifies the finger joints 25 based on characteristics of knuckle creases. In another embodiment, any other methods may be employed to identify the finger joints 25. For example, an outline of the finger is identified via a camera and a position of the finger joint 25 is defined based on relative distance within the finger so as to identify the finger joints 25.

Accordingly, the controller 18 determines the locations of the finger joints 25. In this embodiment, the locations of the finger joints 25 correspond to the positions of the transducer array 30 over the identified finger joints 25 which are identified, via the controller 18, from the recorded positions of the transducer array 30 in scanning the entire hand 24. The controller 18 is configured to control the actuator 32 to move the transducer array 30 according to the locations of the finger joints 25. In this embodiment, the controller 18 outputs control signals causing the actuator 32 to move the transducer array 30 to the identified positions of the transducer array 30. The transducer array 30 is operated in the second imaging mode, such as PDI imaging mode or the high resolution PDI imaging mode, to sweep the finger joints 25 and the imaging device 14 is also operated in the second imaging mode to imaging the blood flow in the finger joints 25 to aid a clinician in identifying disease states, assessing need for treatment, determining suitable treatment options, tracking the progression of the disease, and/or monitoring the effect of the treatment on the disease states. The transducer array 30 can scan the same anatomical locations of the finger joints 25 due to the detected positions of the transducer array 30 so as to facilitate following up treatment effect, monitoring pathological stage progress.

The controller 18 includes a processor or processing unit that receives signals and controls the operation of the transducer array 30, the temperature changing device 36 and the actuator 32. For purposes of this disclosure, the term “processing unit” shall mean a presently developed or future developed processing unit that executes sequences of instructions contained in a memory. Execution of the sequences of instructions causes the processing unit to perform steps such as generating control signals. The instructions may be loaded in a random access memory (RAM) for execution by the processing unit from a read only memory (ROM), a mass storage device, or some other non-transitory persistent storage. In other embodiments, hard wired circuitry may be used in place of or in combination with software instructions to implement the functions described. For example, controller 18 may be embodied as part of one or more application-specific integrated circuits (ASICs). Unless otherwise specifically noted, the controller is not limited to any specific combination of hardware circuitry and software, or to any particular source for the instructions executed by the processing unit.

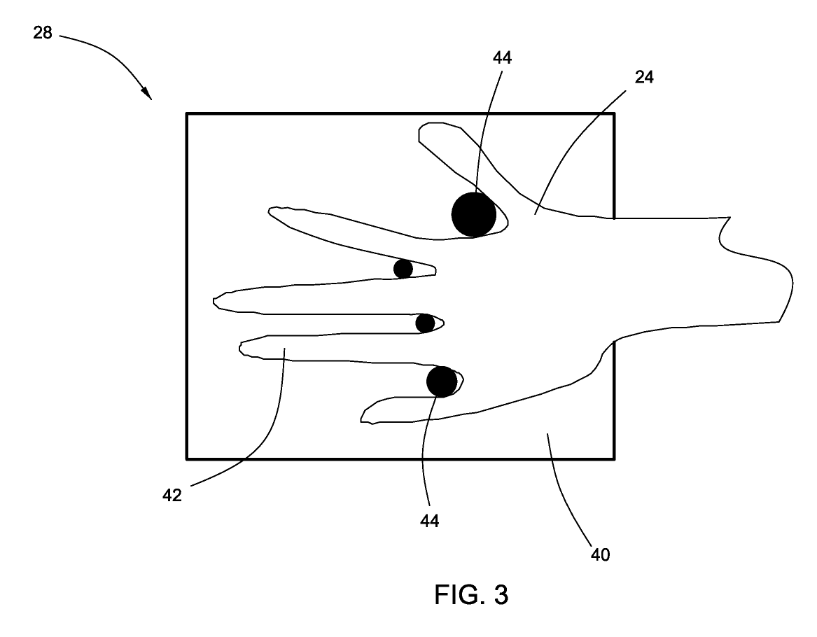

FIG. 3 illustrates a schematic view of a holder 28 according to another embodiment. The holder 28 in FIG. 3 includes a base 40 and multiple finger retainers 44. The base 40 may include a rectangular, semicircular or U-shaped position receiving and supporting the hand 24. The finger retainers 44 are configured to retain fingers 42 of the hand 24 stationary during scanning. In one embodiment, the finger retainers 44 may include posts extending from the base 40 of the holder 28 and located so as to between adjacent fingers 42 to retain the fingers 42 stationary. The posts may be configured and shaped ergonomically. For example, the particular post 44 extending between the thumb and index finger is widened so through ergonomically contact sides of the person's palm and thumb. In another embodiment, the finger retainers 44 may be formed in any other structures being capable of retain the fingers 42 stationary. In another embodiment, the holder 28 may have other configurations.

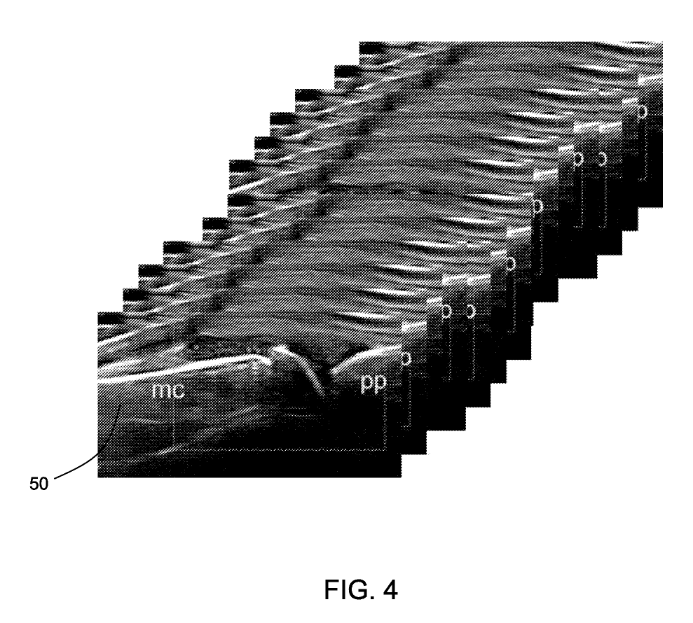

FIG. 4 illustrates 2D ultrasound images 50 of slices of the hand 24 according to an embodiment. The 2D ultrasound images 50 are imaged in the imaging device 14 one by one as the transducer array 30 scanning the hand 24 slice by slice. Each 2D ultrasound image 50 displayed on the screen of the imaging device 14 is grabbed by the screen grabbing device 16 and further received by the controller 18.

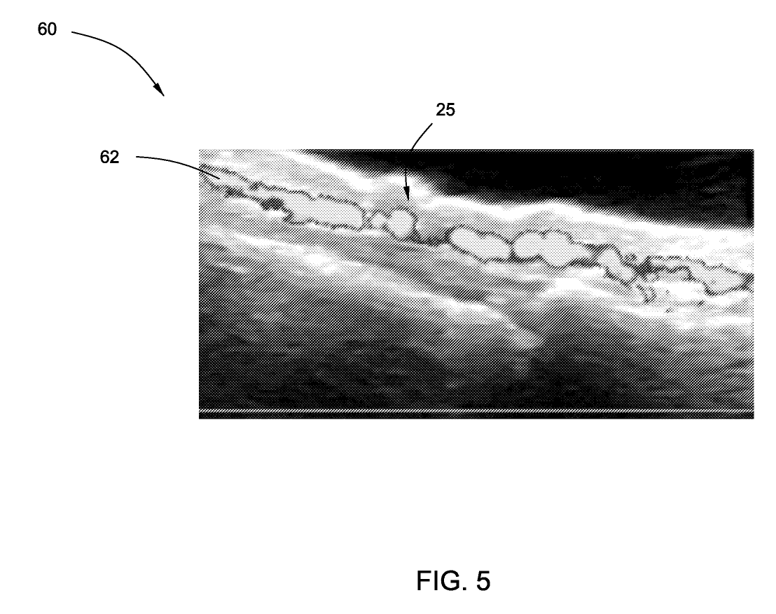

FIG. 5 illustrates an image 60 of a finger joint 25 according to an embodiment. The image 60 shows bloodstream 62 flowing through the finger joint 25 for facilitating detection, quantification and tracking of rheumatoid arthritis. In one embodiment, the bloodstream in the image is marked in color such as red or blue. The imaging device 14 generates multiple images 60 for each finger joint 25 to detect the bloodstream 62 through the slices of the finger joint 25.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A system, comprising:
a tank configured to contain fluid and allow a hand to be immersed in the fluid;
a holder located in the tank and configured to hold the hand;
a transducer array positioned adjacent to the tank and operable in at least two imaging modes; and
an actuator coupled to the transducer array and configured to move the transducer array in the at least two imaging modes.

2. The system of claim 1, further comprising one or more temperature sensors for detecting the temperature of the fluid and a temperature changing device for regulating the temperature of the fluid.

3. The system of claim 2, further comprising a controller coupled to the one or more temperature sensors and the temperature changing device for outputting control signals based on the detected temperature of the fluid, and wherein the temperature changing device is operative to warm or cool the fluid in response to the control signals.

4. The system of claim 1, wherein the holder comprises a plurality of finger retainers configured to retain fingers of the hand.

5. The system of claim 1, further comprising one or more guide rails connected with the transducer array, and wherein the transducer array is moved along the one or more guide rails.

6. The system of claim 5, wherein the one or more guide rails comprise a longitudinal rail, a transverse rail perpendicular to the longitudinal rail and a vertical rail perpendicular to the longitudinal rail and the transverse rail.

7. The system of claim 1, further comprising at least one position sensor for detecting positions of the transducer array.

8. The system of claim 7, further comprising a controller coupled to the at least one position sensor for identifying locations of a plurality of finger joints of the hand and controlling the actuator to move the transducer array according to the locations of the plurality of finger joints.

9. The system of claim 1, further comprising:
an imaging device coupled to the transducer array and configured to generate and display ultrasound images of the hand;
a screen grabbing device coupled to the imaging device and configured to grab a screen of the imaging device to get the ultrasound images from the screen; and
a controller coupled to the screen grabbing device and configured to receive the ultrasound images therefrom and identify locations of a plurality of finger joints of the hand from the ultrasound images.

10. The system of claim 9, wherein the ultrasound images of the hand comprise b-mode ultrasound images of the hand.

11. The system of claim 9, wherein the imaging device is further configured to generate Power Doppler Imaging (PDI) images or high resolution PDI images of the plurality of finger joints.

12. A system, comprising:
a tank configured to contain fluid and allow a hand to be immersed in the fluid;
a holder located in the tank and configured to hold the hand;
a transducer array positioned adjacent to the tank and operable in a first imaging mode and a second imaging mode, the transducer array being configured to scan the hand in the first imaging mode and scan a plurality of finger joints of the hand in the second imaging mode; and
a processing unit coupled to the transducer array and configured to control the transducer array in the first imaging mode and the second imaging mode, the processing unit being configured to generate ultrasound images of the hand in the first imaging mode and generate images of the plurality of finger joints in the second imaging mode.

13. The system of claim 12, wherein further comprising one or more temperature sensors for detecting the temperature of the fluid and a temperature changing device for regulating the temperature of the fluid.

14. The system of claim 13, wherein the processing unit comprises a controller coupled to the one or more temperature sensors and the temperature changing device for outputting control signals based on the detected temperature of the fluid, and wherein the temperature changing device is operative to warm or cool the fluid in response to the control signals.

15. The system of claim 12, wherein the holder comprises a plurality of finger retainers configured to retain fingers of the hand.

16. The system of claim 12, further comprising an actuator connected with the transducer array and one or more guide rails connected with the transducer array, and wherein the transducer array is moved by the actuator along the one or more guide rails.

17. The system of claim 12, further comprising at least one position sensor for detecting positions of the transducer array.

18. The system of claim 12, wherein the processing unit comprises:
an imaging device coupled to the transducer array and configured to generate and display the ultrasound images of the hand;
a screen grabbing device coupled to the imaging device and configured to grab a screen of the imaging device to get the ultrasound images from the screen; and
a controller coupled to the screen grabbing device and configured to receive the ultrasound images therefrom and identify locations of a plurality of finger joints of the hand from the ultrasound images.

19. The system of claim 18, wherein the ultrasound images of the hand comprise b-mode ultrasound images of the hand.

20. The system of claim 18, wherein the imaging device is further configured to generate Power Doppler Imaging (PDI) images or high resolution PDI images of the plurality of finger joints.
